(19) United States
(12) Patent Application Publication
(10) Pub. No.: US 2004/0010268 A1
(43) Pub. Date: Jan. 15, 2004

(54) Dermabrasion/microdermabrasion apparatus

(76) Inventor: Michael A. Gabehart, Anaheim, CA (US)

Correspondence Address: Kit M. Stetina, Esq., STETINA BRUNDA GARRED & BRUCKER, Suite 250, 75 Enterprise, Aliso Viejo, CA, 92656, US

(21) Appl. No.: 10/193,548

(22) Filed: Jul. 11, 2002

(51) Int. Cl. A61B 17/50

(52) U.S. Cl. 606/131

(57) ABSTRACT

A dermabrasion/microdermabrasion apparatus, having a central control unit and a hand piece. Negative and positive pressure is provided to the hand piece from the central control unit. The hand piece further has a sealed chamber, a brush wheel and a turbine. The sealed chamber has an opening and a negative pressure supply port to receive the negative pressure. The brush wheel is installed in the seal chamber and brought in contact with a skin to be treated through the opening during dermabrasion/microdermabrasion. The turbine is installed external to the sealed chamber and driven by the positive pressure to provide a rotational force to the brush wheel during dermabrasion/micro-dermabrasion.

### CROSS REFERENCE OF RELATED APPLICATION

(Not Applicable)

### STATEMENT RE: FEDERALLY SPONSORED RESEARCH/DEVELOPMENT

(Not Applicable)

### BACKGROUND OF THE INVENTION

The present invention relates generally to a dermabrasion/microdermabrasion apparatus. More particularly, the present invention relates to a dermabrasion/microdermabrasion apparatus that applies friction to the skin to perform desired therapeutic treatment.

Currently, the commonly applied dermabrasion technologies include particulate dermabrasion and vacuum assisted mechanical dermabrasion. The particulate dermabrasion uses a medium such as corundum or salt such as aluminum oxide in a sandblasting-like fashion to polish the skin. The major disadvantages of such prior art particulate dermabrasion are that particles can be lodged in the skin and a substantial amount of the aluminum oxide may be left behind on the skin. Though no toxic effects have been shown for the commonly used medium, aluminum oxide has been shown to cause inflammatory changes to the lungs in workers who frequently have inhaled aluminum oxide. Further, the users' eyes must be protected from the highly abrasive dust, which can injure the cornea. Moreover, the particles of the abrasive medium can be left imbedded in the skin surface resulting in long term irration and provide a situs for subsequent bacterial infections.

The vacuum assisted mechanical dermabrasion prior art uses a stainless steel wand coated with synthetic diamonds at the functional tip. The central hollow portion of the wand functions as the source of the vacuum for waste removal. The vacuum assisted mechanical dermabrasion has the disadvantage of offering little room for regulating power and parameters. Therefore, the dermabrasion effect is limited.

### BRIEF SUMMARY OF THE INVENTION

The present invention provides a dermabrasion/microdermabrasion apparatus. The dermabrasion/microdermabrasion apparatus of the present invention comprises a central unit and a hand piece. The central control unit provides a negative pressure and a positive pressure to the hand piece. The hand piece comprises a sealed chamber, a brush wheel and a turbine. The sealed chamber has an opening or aperture to contact the skin to be treated and a negative pressure supply port to receive the negative pressure. By application of the negative pressure, the skin to be treated is maintained in contact with the periphery of the brush wheel installed in the sealed chamber during the treatment. The turbine installed in an external compartment of the hand piece is driven by the positive pressure, so as to translate a rotational force to the brush wheel for dermabrasion/microdermabrasion. Therefore, friction is generated between the skin to be treated and the rotating brush wheel.

The central control unit further comprises a negative pressure adjusting means and a positive pressure adjusting means to control the negative and positive pressure, respectively. The central control unit also comprises an indicator to display the rotational speed of the brush wheel, and an indicator to display the amount of negative pressure supplied to the sealed chamber.

In one embodiment, the hand piece further comprises a handle with a negative pressure channel and a positive pressure channel to connect to the central control unit via an extension tube. A primary filter is installed between the hand piece and the central control unit, while a secondary filter is installed in the central control unit to prevent contamination.

By bringing the brush wheel in contact with the skin, a friction is generated at the interface of the skin to the periphery of the brush wheel. The amount of friction is controlled by the negative pressure and the rotational speed of the brush wheel which thereby controls the removal rate of the stratrum corneum of the skin.

The present invention further provides a method of dermabrasion/microdermabrasion including the step of positioning the opening of the sealed chamber upon the skin to be treated; applying a vacuum to the sealed chamber such that the skin is maintained in contact with the brush wheel; rotating the brush wheel to generate friction between the skin and the brush wheel to remove a part of the skin; and moving the hand piece around the skin to be treated so that the stratrum corneum in various part of the skin is thoroughly removed.

The present invention performs dermabrasion/microdermabrasion treatment by applying friction between the skin and the face. The drawbacks occurring to the particulate polishing method are eliminated. Further, the limitation for the vacuum-assisted dermabrasion is also resolved.

### BRIEF DESCRIPTION OF THE DRAWINGS

These, as well as other features of the present invention, will become more apparent upon reference to the drawings wherein:

FIG. 1 shows application of the dermabrasion/microdermabrasion apparatus on facial skin of the user;

FIG. 2 schematically shows the external structure of the dermabrasion/microdermabrasion apparatus;

FIG. 3 is an exploded cross sectional views schematically showing the internal components of hand piece of the dermabrasion/microdermabrasion apparatus;

FIG. 4 shows cross sectional view of the vacuum chamber with a brush wheel applied to skin; and

FIG. 5 is an exploded perspective view of the hand piece of the dermabrasion/microdermabrasion apparatus.

### DETAILED DESCRIPTION OF THE INVENTION

By way of overview, the dermabrasion/microdermabrasion apparatus of the present invention incorporates a brush wheel which is applied to an outer layer of the skin to be treated, such that the non-living epithelial dermis, also known as the stratum corneum, is removed. According to specific condition of the skin, various materials and configurations of the brush wheel can be selected. The brush wheel is housed in a sealed chamber and driven by a turbine to treat the skin. By rotating the brush wheel and supplying a negative pressure, i.e., vacuum, to the sealed chamber, the skin is pulled into contact with periphery of the brush wheel, and friction between the brush wheel and the skin effectively removes the stratum corneum of the skin. The rotational speed of the turbine can be adjusted manually or automatically according to the conditions of the skin.

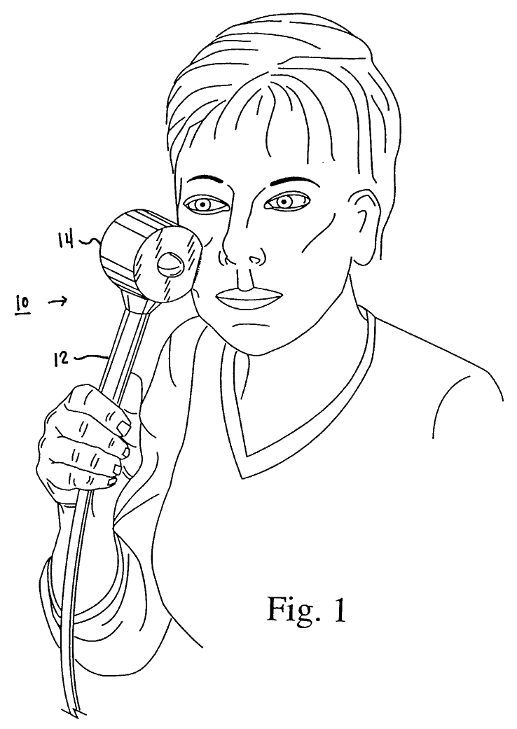

FIG. 1 shows the dermabrasion/microdermabrasion treatment performed on a facial skin using the dermabrasion/microdermabrasion apparatus provided by the present invention. As shown in FIG. 1, the dermabrasion/microdermabrasion apparatus comprises a hand piece 10 having a handle 12 and an application head 14. While performing dermabrasion/microdermabrasion treatment, the user or the operator can simply hold and move the handle 12 with the application head 14 placed on the skin to be treated.

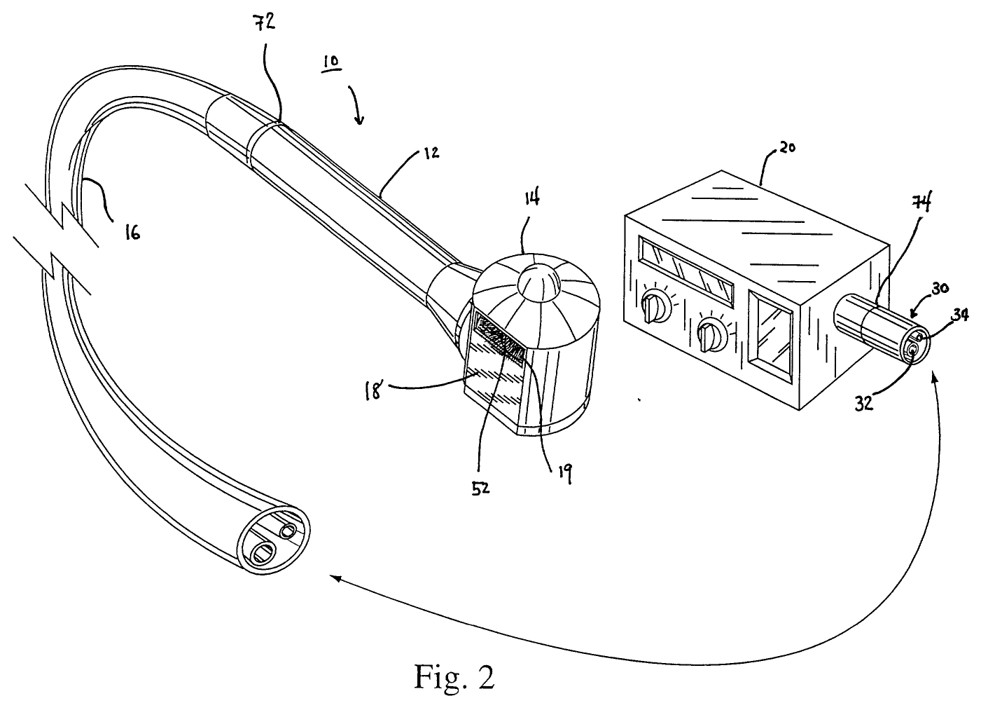

As shown in FIG. 2, the dermabrasion/microdermabrasion apparatus comprises a central control unit 20 which is connected to the hand piece 10 via a flexible conduit or an extension tube 16. The central control unit 20 houses conventional pneumatic pumps to provide both a positive and negative pressure source. The output of the pressure sources communicate with a combination port 30, which encloses a positive pressure port 32 and a negative pressure port 34 to provide positive and negative pressure, respectively. The magnitude of the positive and negative pressure can be controlled automatically according to the condition of the skin to be treated or manually by tuning positive and negative pressure control knobs 26 and 28.

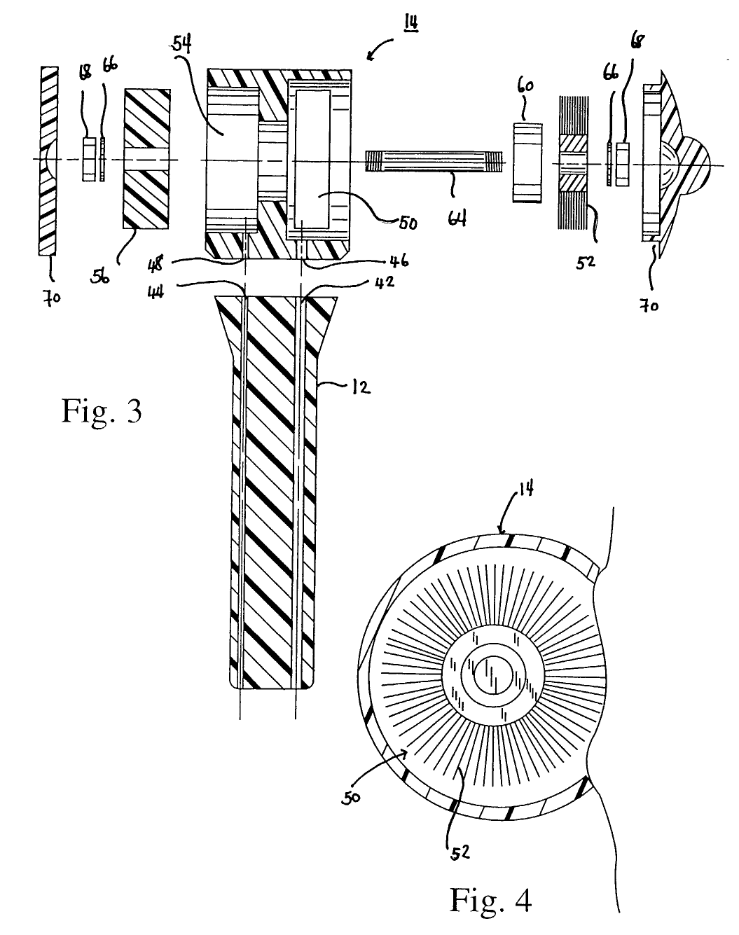

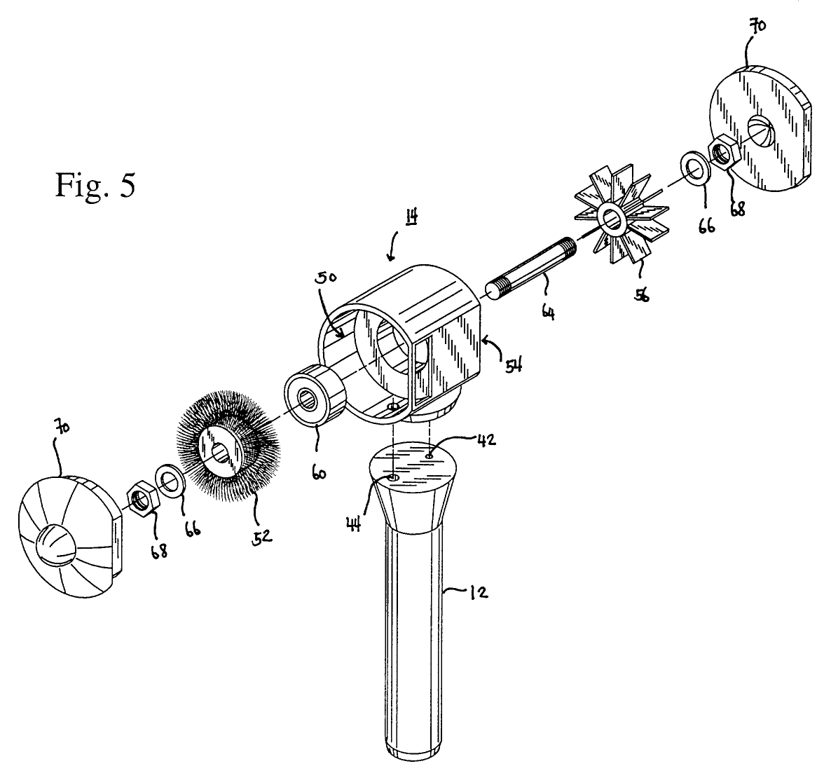

The application head 14 is substantially cylindrical, yet a portion of the surface area 18 is provided with a flat surface. The flat surface allows direct contact between the application head 14 and the skin to be treated. The flat surface area 18 includes an opening or aperture 19, which allows contact between a brush wheel 52 (referring FIGS. 3 and 5) and the skin to be treated. Further, the brush wheel 52 can be easily replaced through the aperture 19.

FIG. 3 illustrates a cross sectional view of the hand piece 10, while FIG. 5 shows an exploded view of the hand piece 10. Referring to FIG. 3, the handle 12 of the hand piece 10 includes a positive pressure supply channel 42 and a negative pressure supply channel 44 therein. The application head 14 comprises a chamber 50, in which a brush wheel 52 is disposed, and a compartment 54 external to the chamber 50, in which a turbine 56 for driving the brush wheel 52 is installed. The chamber 50 and the compartment 54 are separate or sealed from each other by a central wall which mounts a rotary bearing 60. The brush wheel 52 and the turbine 56 are supported by a common central axle 64 extending into both the chamber 50 and the compartment 54 through the bearing 60. Washers 66 and nuts 68 are engaged with threads formed at opposite ends of the central axle 64, such that the brush wheel 52 and the turbine 56 can be fixed and positioned as required. At each end of the chamber 50 and the compartment 54, a cap 70 is used to seal the chamber 50 and the compartment 54. Preferably, one of the caps 70 has an dome shape and a semi-spherical center protruding outwardly, while the other has an internal recess to firmly emplace the central axle 64.

As best shown in FIG. 3, the negative pressure port 48 is formed in the wall of the chamber 50 and is aligned and connected with the negative pressure supply channel 44 of the handle 12. The positive pressure port 46 is formed in the wall of the compartment 54 to align and connect to the positive pressure supply channel 42. Thereby, the negative and positive pressure supplied from the central control unit 20 can be provided to the chamber 50 and the compartment 54, respectively via the flexible conduit 16. The above components in the hand piece 10 as shown in FIG. 3 and FIG. 5 are only schematical and exemplary structures.

In addition, a primary filter 72 may be mounted at the back of the hand piece 10, that is, between the handle 12 and the extension tube 16, and a secondary filter 74 can be installed in the central control unit 20 to prevent contamination to the pneumatic pump and the control unit.

With the structure defined, the operation of the present invention can be described. FIG. 4 shows the cross sectional view of the application piece 14 applied to the skin. As mentioned above, a negative pressure, preferably a vacuum force is applied to the chamber 50 during dermabrasion/micro-dermabrasion. By placing the application piece 14 with the opening 19 positioned on the skin to be treated, the skin is pulled upwardly into the chamber 50 through the opening 19; therefore, a direct contact between the brush wheel 52 and the skin is maintained. By rotating the brush wheel 52, friction between the periphery of the brush wheel 52 and the skin removes the outer layer of the skin. Again, according to specific skin condition, the specific material and shape, i.e., dimension and width, of the brush wheel can be varied.

In addition to the configuration and material of the brush wheel, the negative pressure applied to the chamber 50 and the positive pressure applied to the compartment 54 to drive the turbine are two major factors that directly affect the magnitude of the applied friction. In one embodiment of the present invention, the rotational speed, i.e., the revolution per minute (RPM) of the brush wheel 56 can be monitored by an indicator 22, while the negative pressure supplied to the sealed chamber 50 can be monitor by another indicator 24. The indicators 22 and 24 includes LED light bars or other illumination devices installed at a panel of the central control unit 20.

By moving the opening 19 of the application head 14 around the skin to be treated, the dead cells of the outer layer the skin can be removed. In the above embodiment, the opening 19 has a rectangular shape. It will be appreciated that those skilled in the art may calculate a precise angle or configuration shape to allow an optimal contact between the brush wheel and the skin without exceeding the spirit and scope of the present invention.

Further, those skilled in the art will recognize that the dermabrasion/microdermabrasion apparatus provided by the present invention can effectively remove the stratrum corneum without damaging other portion of the skin. In fact, an increase in capillary circulation caused by the vacuum and mechanical manipulation of the dermis benefits the effect on the dermis. Moreover, as the brush wheel can be easily detached and attached, the dermabrasion/microdermabrasion apparatus can be easily cleaned and sterilized.

In the above embodiment, the dermabrasion/microdermabrasion is performed on a dry skin. It is appreciated that the dermabrasion/microdermabrasion apparatus provided by the present invention is also applicable to a wet skin. Further, the friction between the skin and the brush wheel may be increased or decreased by a fluid applied on the skin. The fluid may be supplied from other source, or by incorporating an additional fluid wash irrigator in the dermabrasion/microdermabrasion apparatus.

Indeed, each of the features and embodiments described herein can be used by itself, or in combination with one or more of other features and embodiment. Thus, the invention is not limited by the illustrated embodiment but is to be defined by the following claims when read in the broadest reasonable manner to preserve the validity of the claims.

What is claimed is:

1. A dermabrasion/microdermabrasion apparatus, comprising:
a central control unit, to provide a negative pressure and a positive pressure; and
a hand piece, further comprising
a sealed chamber, having an opening and a negative pressure supply port to receive the negative pressure;
a brush wheel, installed in the sealed chamber, the periphery of said brush wheel being juxtapositioned to bring in contact with a skin to be treated through the opening during dermabrasion/microdermabrasion; and
a turbine, external to the sealed chamber and driven by the positive pressure to translate a rotational motion to the brush wheel during dermabrasion/micro-dermabrasion.

2. The dermabrasion/microdermabrasion apparatus according to claim 1, wherein the central control unit further comprises a negative pressure adjusting means and a positive pressure adjusting means to control the negative and positive pressure, respectively.

3. The dermabrasion/microdermabrasion apparatus according to claim 1, wherein the central control unit further comprises an indicator to display the rotational speed of the brush wheel.

4. The dermabrasion/microdermabrasion apparatus according to claim 1, wherein the central control unit further comprises an indicator to display the negative pressure supplied to the sealed chamber.

5. The dermabrasion/microdermabrasion apparatus according to claim 1, wherein hand piece further comprises a compartment external to the sealed chamber to enclose the turbine therein.

6. The dermabrasion/microdermabrasion apparatus according to claim 5, wherein the compartment comprises a positive pressure port to receive the positive pressure.

7. The dermabrasion/microdermabrasion apparatus according to claim 1, wherein the hand piece further comprises a handle with a negative pressure channel and a positive pressure channel to connect to the central control unit.

8. The dermabrasion/microdermabrasion apparatus according to claim 1, further comprising an extension tube connected between the hand piece and the central control unit.

9. The dermabrasion/microdermabrasion apparatus according to claim 1, further comprising a primary filter installed between the hand piece and the central control unit.

10. The dermabrasion/microdermabrasion apparatus according to claim 1, further comprising a secondary filter installed in the central control unit.

11. A dermabrasion/microdermabrasion apparatus, comprising a brush wheel enclosed in a sealed chamber which receives a negative pressure to bring a skin to be treated in contact with the brush wheel through an opening in the sealed chamber, wherein the brush wheel is rotating during dermabrasion/microdermabrasion to generate friction between the periphery of the brush wheel and the skin.

12. The dermabrasion/dermabrasion apparatus according to claim 11, further comprising a turbine external to the sealed chamber to drive the brush wheel.

13. The dermabrasion/microdermabrasion apparatus according to claim 11, further comprising a central control unit to control the negative pressure and the rotational speed of the brush wheel.

14. A method of dermabrasion/microdermabrasion, comprising:
providing a sealed chamber having an opening and a brush wheel disposed therein;
positioning the opening on the skin to be treated;
applying a vacuum to the sealed chamber such that the skin is pulled into the opening to contact the brush wheel; and
rotating the brush wheel to generate frictional contact between the skin and the brush wheel to remove a part of the skin.

15. The method according to claim 14, further comprising a step of exhausting the removed part of the skin by vacuum from the sealed chamber.

16. A dermabrasion/microdermabrasion apparatus that applies a friction to a skin to be treated and remove a part of the skin by applying a negative pressure to the skin.
